(19) United States
(12) Patent Application Publication
(10) Pub. No.: US 2023/0389793 A1
(43) Pub. Date: Dec. 7, 2023

(54) METHOD FOR ASSIGNING A VERTIGO PATIENT TO A MEDICAL SPECIALTY

(71) Applicant: Vertify GmbH, Graz (AT)

(72) Inventor: Christof STOCKER, Güssing (AT)

(73) Assignee: Vertify GmbH, Graz (AT)

(21) Appl. No.: 18/032,824

(22) PCT Filed: Oct. 19, 2021
(86) PCT No.: PCT/AT2021/060388

§ 371 Date: Apr. 20, 2023

(30) Foreign Application Priority Data
Oct. 20, 2020 (AT) A236/2020

(51) Int. Cl. A61B 3/113; A61B 5/11; A61B 5/00

(52) U.S. Cl. CPC A61B 3/113; A61B 5/1128; A61B 5/7267

(57) ABSTRACT

The present invention relates to a method for assigning a dizzy patient (SP) to a medical specialty (MF), comprising the following steps:
Capture of eye movements (AB) of the dizzy patient (SP) in the form of video data (VD),
Processing the acquired video data (VD) in a neural network (NN),
Determine at least one medical specialty (MF) based on the result of processing in the neural network (NN),
Outputting an assignment of the dizzy patient (SP) to the specific at least one medical specialty (MF).

The present invention relates to a method for assigning a patient with dizziness or vertigo to a medical specialty, a training method for training a neural network for use in such a method, an assignment system for performing such a method, and a corresponding computer program product.

It is well known that patients visit their general practitioner for a wide variety of reasons. Next to headaches, the second most common reason for visiting the general practitioner is vertigo or dizziness. However, vertigo symptoms can have a wide variety of causes. Therefore, a wide variety of specialists are needed to diagnose the cause of a vertigo symptom. For example, it is possible that vertigo has a neurological cause. Accordingly, a neurological cause would need to be investigated by a neurological specialist. It is also possible that vertigo stems from a disorder in the inner ear. In such a case, the necessary specialist to diagnose the actual cause would be an ear, nose and throat specialist.

With the known solutions, an assignment and thus a referral to the respective specialist will be made exclusively on the basis of a time-limited examination by the general practitioner. Often, this can lead to patients being sent to a number of different specialists, who either successively or even in parallel try to diagnose the cause of the vertigo. This leads to a high expenditure of time for the specialists as well as for the patient. In addition, this leads to high costs, since there is often only one cause for the vertigo. This in turn means that in many cases only one of the specialists is actually able to diagnose the cause of the vertigo, while the other specialists consulted are unable to make a successful diagnosis. Further, there may be unnecessary use of expensive imaging modalities, such as magnetic resonance imaging. The increased cost and effort here is borne either by the patient himself or by society in the form of the respective health insurance.

It is the task of the present invention to at least partially overcome the disadvantages described above. In particular, it is the task of the present invention to achieve assignment to an explicit medical specialty in a cost-effective and simple manner.

The foregoing task is solved by a method having the features of claim 1, a training method having the features of claim 12, an assignment system having the features of claim 15, and a computer program product having the features of claim 17. Further features and details of the invention result from the subclaims, the description and the drawings. Features and details described in connection with the method according to the invention naturally also apply in connection with the training method according to the invention, the assignment system according to the invention and the computer program product according to the invention, and vice versa in each case, so that reference is or can always be made mutually to the individual aspects of the invention with respect to the disclosure.

According to the invention, the method serves to assign a patient suffering from vertigo to a medical specialty. For this purpose, such a method has the following steps:
Capture of eye movements of the dizzy patient in the form of video data,
Processing the captured video data in a neural network,
Determining at least one medical specialty based on the result of processing in the neural network,
Issuing an assignment of the dizzy patient to the specific, at least one medical specialty.

Thus, a method according to the invention serves to find out a medical specialty which is able to diagnose with high probability the actual cause of the vertigo for the dizzy patient. In this context, a dizzy patient in the sense of the present invention is a patient who suffers from a vertigo/dizziness symptom. A method according to the present invention now allows the assignment of a medical specialty to be provided independently of or in addition to the experience of the respective general practitioner. The assigned and issued medical specialty is thereby with high probability that specialty in which the cause of the vertigo symptom that is yet to be diagnosed lies, so that through the assignment to the medical specialty, that specialist can be consulted directly who can also diagnose the cause of the vertigo with high probability. Furthermore, in the case of multiple referrals according to the state of the art, a possibly reduced sense of responsibility of the individual medical specialists can be assumed. In the context of the present invention, a neural network means any form of artificial intelligence, including, for example, any form of differentiable programming.

Compared to the previous solutions, it is no longer necessary to consult a large number of specialists in parallel and/or one after the other, but only one specialist or a few specialists who correspond to the assigned medical specialty can be consulted. As a result, significantly less time is required to consult the specialist with the patient and unnecessary consultations with other specialists can be avoided. The reduced time required is also accompanied by reduced costs, as unnecessary visits to specialists no longer have to be paid for.

A method according to the invention is based on the fact that eye movements of the patient suffering from vertigo are recorded. This is done with a video sensor, for example in the form of a camera system. A particularly simple and inexpensive embodiment of a method according to the invention, as explained below, can be carried out on a mobile terminal. However, it is of course also possible that special terminal devices, which have associated video sensors or camera systems, may be installed at preferably a GP. In the simplest way, however, a method according to the invention can run as a software product or as a computer program product, for example in the form of an application, on a mobile terminal device in the form of a tablet or a mobile radio device.

In principle, it is also possible for the eye movements to be recorded separately from the method according to the invention. For example, existing camera systems can record the eye movements in the form of a video file and make it available to the method according to the invention for further processing.

Using the smartphone as an example, it will now be explained how a method according to the invention can be carried out. With the aid of the camera of the smartphone, it is possible to record the eye movements of the patient with vertigo in the form of video data. These eye movements can be freely recorded eye movements or guided eye movements. As will be explained later, guided eye movements are particularly advantageous, and can be recorded on the basis of preferably standardized eye movement tests. For example, eye movement patterns with minute movements and/or twitches can allow conclusions to be drawn about the relevant medical specialties.

Once the eye movements of the dizzy patient have been captured in the form of video data, they can be processed with the aid of a neural network. For the purposes of the present invention, a neural network is to be understood as the use of a so-called artificial intelligence. In this context, the neural network is a trained neural network which is provided with corresponding neural network nodes and associated weights on the basis of a plurality of eye movement data labeled in a controlled manner. This leads to the fact that the processing of the captured video data in this trained neural network makes it possible that the captured video data can be assigned with a high degree of certainty to a medical specialty, on which in a subsequent consultation of the associated specialist also a diagnosis of the cause of vertigo can be made. In other words, the video data are processed in the neural network, whereby a determination of at least one medical specialty is made by the neural network on the basis of the result of this processing.

Finally, in a method according to the invention, the output of this assignment of the dizzy patient to the specific, at least one, medical specialty takes place. It is therefore possible that after the recording of the eye movements and the processing in the neural network, an output of one or even more medical specialties is possible directly on the mobile terminal, which with a high probability subsequently to a procedure according to the invention allow the diagnosis of the cause of the vertigo. The assigned medical specialty thus leads to the dizzy patient subsequently visiting the specialist of the assigned medical specialty, where a diagnosis of the actual cause of the vertigo and possibly an initiation of a suitable therapy can be made.

As can be seen on the basis of the preceding explanation, the assignment to the medical specialty is now at least partially normalized. This is based on the fact that the experience of a large number of experts is stored in the neural network by the corresponding training procedures in the form of the weighting of the individual network nodes. The recorded eye movements in the form of the video data alone or in combination with patent answers explained later can thus automatically and with high accuracy assign the medical specialty in which the cause of the vertigo can be diagnosed with high probability.

It should also be noted that a procedure according to the invention does not serve to diagnose a cause, but rather enables a diagnosis to be made following the procedure according to the invention. Whereas in known procedures a general practitioner selected a large number of specialists who had to carry out all diagnostic attempts for the cause of the vertigo, in the manner according to the invention it is now possible to explicitly select one or significantly fewer specialists in the area of the assigned medical specialties in order to now carry out the diagnosis of the cause of the vertigo more cost-effectively, more quickly and, above all, more simply afterwards. Sending to the wrong medical specialties is avoided in this way, or at least the probability is significantly reduced. Regarding the training and the design of the neural network, more detailed explanations will follow later.

There may be advantages if, in a method according to the invention, the video data are anonymized before processing in the neural network, in particular translated into eye movement parameters. While it is in principle possible to directly process video data in a neural network, the translation into anonymized data, for example in the form of eye movement parameters, can bring additional advantages. On the one hand, it becomes possible in this way to provide a compression of the amount of data, so that the anonymized data have a significantly smaller memory size than this applies to the recorded video data. At the same time, it becomes possible in this way to provide anonymization, which subsequently allows these anonymized eye movements to be transmitted from the mobile terminal to other devices. This is particularly advantageous if, for example, the neural network is centralized in a network solution, for example in the form of a processing cloud. The processing and use of personal data in a cross-system network is fundamentally supported by high security standards. Thus, the anonymizing step allows anonymized eye movement information to be communicated to a central network without security concerns, while patient-related data remains on the mobile device. Among anonymized eye movement parameters, directional vectors of the individual eyes, in particular in the form of the gaze direction of the eyes, in the form of a head tilt and/or in the form of a head orientation, are conceivable, for example. However, it is also possible that the eye movement parameters are stored in another form, such as an Euler angle, rotation matrix, or pixel translation. It is also conceivable that other supporting information, such as head tilt and/or head orientation as direction vectors, rotation matrix, or quaternions, are included. The anonymity of these data is given by the low sampling rate as well as missing biometric information, such as the interpupillary distance. The step of anonymization is preferably performed locally in the respective acquisition device, so that the separation between patient-related data and anonymized data is provided here in a local manner. This translation can be provided, for example, in the form of an algorithm and/or likewise by the use of one or more neural networks.

There are further advantages if, in a method according to the invention, the acquired video data are transmitted, in particular in anonymized form, from an acquisition device to a processing device in which the processing is carried out by means of the neural network, preferably with the specific medical specialty being transmitted back to the acquisition device and output at the acquisition device by means of an output device. As has already been explained in the preceding paragraph, there may be advantages in providing the processing intelligence in the form of the processing neural network centrally on a server, for example in a cloud. In particular, if the eye movements in the form of the video data are anonymized, for example by the eye movement parameters explained in the preceding paragraph, this anonymized data can be transmitted, preferably wirelessly, to this central cloud server and processed there. If the server is a suitably secured variant, non-anonymized data can also be stored and/or processed there. This allows even computationally intensive solutions in the form of the neural network to be displayed centrally on a server, so that the computing capacity does not have to be provided by the mobile terminal. The process here is similar to a speech recognition system based on a server, for example in a cloud. The eye movements are recorded and, if necessary, preprocessed locally with the aid of the acquisition device, processed centrally with the aid of the processing device located on the cloud server, and transmitted back for output to the output device, which is designed in particular integrally with the acquisition device.

Further advantages can be achieved if, in a method according to the invention, at least one test video is played and/or instructions are displayed on a display device of an acquisition device during the recording of eye movements. For example, oculomotor tests such as gaze direction nystagmus, optokinetic nystagmus, gaze sequence movement, fixation suppression can be displayed in the form of visual or audio material using a smart device. For example, such a test video may have a guidance function for a forced or guided eye movement, respectively. It is also possible for the test video to display different light-dark sequences (e.g., replication of a nystagmus drum using a black-and-white bar pattern) in order to elicit corresponding eye reactions in the form of eye movements. In this context, the individual test videos are to be understood in particular as completed eye movement tests, with a separate eye movement being recorded in the form of video data preferably for each eye movement test. The processing in the subsequent step can thereby be specific to the respective eye movement test, for example. For example, if a guided eye movement is performed, the recorded eye movements are to be fed to a first neural network for processing, while in the case of a light-dark variation as an eye movement test, the recorded eye movements are fed to a second, independent neural network. Here it can be seen well that in a simple and inexpensive way not only one but several different eye movement tests can be combined with a method according to the invention, so that by the combined evaluation in correspondingly specifically assigned neural networks, the certainty of assignment to the correct medical specialty and thus the advantages of the present invention can be further improved. It is also conceivable that, in addition to the assignment, supplementary information is output which, for example, can provide focal points for the subsequent diagnosis by the specialist.

It is also advantageous if, in addition to the video data, at least one patient response to at least one patient question is recorded in a method according to the invention. It is thus possible for one or more patient questions to be displayed on the acquisition device at a corresponding display section. In the simplest case, this is a query for basic patient information, such as age, gender, weight, or the like. However, it is also conceivable that a query is made for information relevant to the vertigo system, for example, in what context vertigo occurs, since when the vertigo has occurred, whether there has been a new eyeglass prescription, or the like. The associated patient responses to the patient questions can be used to improve different steps of the procedure. For example, they can be used to select different eye movement tests based on the patient responses. It is also possible to change weights in the neural network processing based on the patient response. Furthermore, it is still possible to correlate the processing result of the neural network with the received patient responses and in this way further increase the certainty of assignment to the correct medical specialty. In this case, the patient questions and the patient answers preferably remain locally on the acquisition device and are in particular not transmitted to the neural network of the processing device or are transmitted only in an anonymized manner.

Further advantages are achievable if, in a method according to the invention, the eye movements are recorded for at least two different eye movement tests, with the video data for different eye movement tests being processed in different neural networks. As has been explained, different eye movement tests may provide different significant information as to which medical specialty is able to diagnose the cause of vertigo. In this embodiment, different eye movements are stored in different video data specific to each of the eye movement tests performed and are accordingly specifically fed to different neural networks. In other words, each neural network outputs its own processing result, which can then be combined into a common processing result and output in the form of a medical specialty assignment. The individual video data for the eye movement tests preferably remain separate from one another and, in particular, are not mixed with one another.

It can also be advantageous if, in a method according to the invention, a quality control of the video data is carried out before and/or during and/or after the recording of the eye movements in the form of video data. By a quality control is to be understood, for example, the alignment of the video sensor, i.e., it is to be checked whether the eyes and the direction of gaze of the dizzy patients are actually detected. Ambient parameters, such as the lighting situation and light alignment, can also be monitored. The quality of the recording itself, in particular in the form of shaking of the video data, can also be monitored. In particular, sensors are used which are already present in the mobile terminal in the form of the acquisition device. The quality requirements and the monitoring result can, on the one hand, lead to the need to perform the eye movement test again. It is also possible to use live feedback to inform the user directly during the eye movement test that the necessary quality requirements are not currently being met, for example that the eyes are not completely within the detection range of the camera, so that the user can adapt the quality criteria during the test.

Further advantages may be gained if, in a method according to the invention, the determination of the at least one medical specialty includes a safety factor which includes the accuracy of the assignment of the at least one medical specialty. This is particularly useful when two or more eye movement tests can be evaluated. In combination, it is thus possible to output the accuracy in a numerical value in the form of the safety factor, which is indicated in particular as a percentage. For example, it can be output that with a high degree of probability a diagnosis will be possible in the medical field of the ear, nose and throat specialist. The safety factor, for example in the form of a confidence factor, for this can be 90 percent, for example. In addition, it can be output that a cause in a neurological field is also possible in principle, but with a reduced probability of, for example, 30 percent. This makes it possible, in the event that an evaluation of the assignment is desired, to provide the dizzy patient or the user of a method according to the invention with additional information. This safety factor can thereby also be assigned to individual eye movement tests, so that the informative value of the assignment of medical specialties can be further increased.

It is also advantageous if, in a method according to the invention, the captured video data are made available for manual review. Such a manual review can be performed, for example, by experts who are able to interpret the eye movements in a manual manner. The method according to the invention is now able to make this captured video data available to this manual review, in particular in video form. This will be particularly useful if the neural network does not allow assignment to a medical specialty with sufficient certainty. The manual review results in accuracy being provided with a high degree of certainty in these individual cases and, moreover, can allow feedback to the neural network so that, in addition, a training effect can be achieved for the individual weighting parameters in the neural network. For example, the method according to the invention can make the acquired video data available for manual review depending on a threshold value for said security factor. For example, results of processing from the neural network with a safety factor of, for example, less than 60 percent can be transmitted to an expert, who can then perform and report back a manual review in the context of telemedicine. This is of course also possible at the direct active request of the user.

There are also advantages if, in a method according to the preceding paragraph, the video data are made available for manual inspection in anonymized form, in particular as artificial video data. While in principle a transmission of the real eye movements in the form of real recorded video data is possible, an anonymized transmission may be desired for the reasons of data protection already mentioned. While the neural network can work with mathematical input information in the form of anonymized eye movements, for example in the form of eye movement parameters, usually experts rely on video information for manual verification. Thus, in this embodiment, it is now possible to generate artificial video data based on the video data in an anonymized manner, which transfers the real recorded eye movements to a virtual and thus artificial head or artificial eyes. In other words, the expert is provided with a digitized and thus virtual eye movement from a real eye movement on an artificial and thus virtual head for the manual examination, so that he can perform a manual examination qualitatively and also quantitatively, but does not receive any personal data of the dizzy patient.

It is also advantageous if additional secondary parameters are recorded in a method according to the invention, in particular at least one of the following:
brightness,
Lighting situation,
Acceleration of an acquisition device,
positioning of an acquisition device.

The above list is not exhaustive. Of course, two or more secondary parameters can also be combined. The use of secondary parameters serves in particular to perform a quality check in order to verify a quality criterion during or after the recording of the eye movements before processing in the neural network. However, this additional information can also be used to subsequently make the processing in the neural network even more accurate, for example, the brightness of the background or environmental parameters such as the ambient temperature when the test is performed. For example, it is possible that eye movements are worse when looking at a bright background and/or when ambient temperatures are particularly low. By recording the corresponding parameters, this can be taken into account during the evaluation. In particular, existing sensors of the acquisition device are used as corresponding sensors. If the acquisition device is a mobile terminal, for example, a gyroscope, an acceleration sensor, a lidar, a brightness sensor or similar sensors can be used.

Also an object of the present invention is a training method for training a neural network for use in a method according to the invention, comprising the following steps:
To provide a variety of eye movements of dizzy patients in the form of video data,
manual labeling of the variety of eye movements,
Training the neural network with the multitude of manually labeled eye movements.

In particular, in addition to conspicuous eye movements of dizzy patients, healthy eye movements are provided for a training procedure. Manual labeling means that eye movements are manually classified into medically conspicuous eye movements or medically inconspicuous eye movements and a medical specialty is assigned. This manual labelling is done by experts who, based on the results from at least one eye movement test performed, can select with a high degree of certainty the relevant medical specialty in which the cause of vertigo can be diagnosed. A large number of such labeled eye movements are now used to train the neural network in the known way. The training effect leads to the fact that in a single network node of the neural network the weights of the corresponding mathematical operation are varied and refined until the output of the neural network corresponds to the desired result in the form of the labeled eye movement. This requires a large amount of eye movement data, which is provided in particular on the basis of existing patient information. However, it is also possible that already anonymized data is used here, which is then used as the basis for the training procedure. Manual labeling also means, for example, the superordinate labeling of an entire group of eye movements that are identified as conspicuous in a superordinate step.

It is further advantageous if artificial video data are generated in a training method according to the invention for making available, which are manipulated in particular manually and/or automatically. These artificial video data can be generated completely artificially. However, it is also conceivable that the artificial video data are generated on the basis of real video data and then additionally manipulated. For example, if the eye movement of a dizzy patient is recorded and labelled, anonymization and translation into artificial video data can be performed from this one recording. If now not only one virtual head is used, but for example ten different virtual heads, the number of ten different artificial video data can be generated from a single recording of a dizzy patient, which can thus be automatically multiplied and provided in higher numbers to the training of the neural network. Despite a low label effort, a high number of training data can be achieved in a short time.

Further advantages can be gained if, in a training method according to the invention, the plurality of eye movements are at least partially labelled multiple times. This means that the same eye movements of the same dizzy patient are labelled several times by different experts in order to avoid an undesired inaccuracy in the neural network. Thereby, over the duration of the labelling process and the training process, the statements and thus the label results of the individual experts can be weighted differently in order to exert different training effects on the neural network according to the different weighting. For example, information such as interagreement between different experts and intraagreement of a single expert could be included in the weighting.

Another object of the present invention is to provide an assignment system for assigning dizzy patients to a medical specialty. Such an assignment system comprises an acquisition device for capturing eye movements of the dizzy patient in the form of video data. Further, a processing device is provided for processing the acquired video data in a neural network. With the aid of a determination device, a determination of at least one medical specialty is possible based on the result of the processings in the neural network. Furthermore, the assignment system comprises an output device for outputting an assignment of dizzy patients to the determined at least one medical specialty. In this regard, the acquisition device, the processing device, the determination device, and/or the output device are particularly adapted to perform a method according to the invention. Thus, an assignment system according to the invention brings the same advantages as have been explained in detail with reference to a method according to the invention.

Advantages may be gained if, in an assignment system according to the invention, the acquisition device and the output device are arranged in a local assignment device and at least the processing device is arranged in a central assignment device. This is to be understood as the cloud solution already explained several times, wherein the eye movements are detected by a mobile terminal device in the form of, for example, a tablet, mobile radio device, digital eyeglass system, digital contact lenses or other digital visual aids as an acquisition device and subsequently transmitted, in particular in an anonymized manner, to the cloud and the processing device arranged there. After the cloud-based processing has been carried out, the processing result is transmitted back to the local assignment device and thus to the mobile terminal, so that it can be output there to the dizzy patient or the person carrying out the test.

Also an object of the present invention is a computer program product comprising instructions which cause an assignment system according to the invention to perform the method steps according to a method according to the invention. Thus, a computer program product according to the invention provides the same advantages as have been explained in detail with reference to an assignment system according to the invention as well as a method according to the invention.

Further advantages, features and details of the invention will be apparent from the following description, in which embodiments of the invention are described in detail with reference to the drawings. In this connection, the features mentioned in the claims and in the description may each be essential to the invention individually or in any combination. It schematically shows:

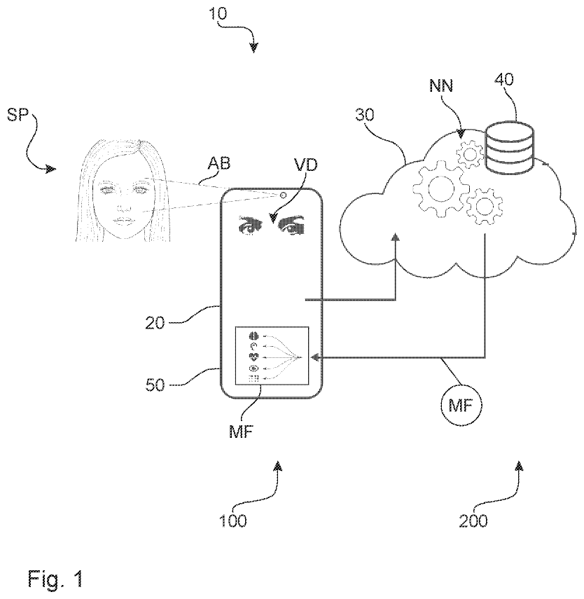

FIG. 1 an embodiment of an assignment system according to the invention,

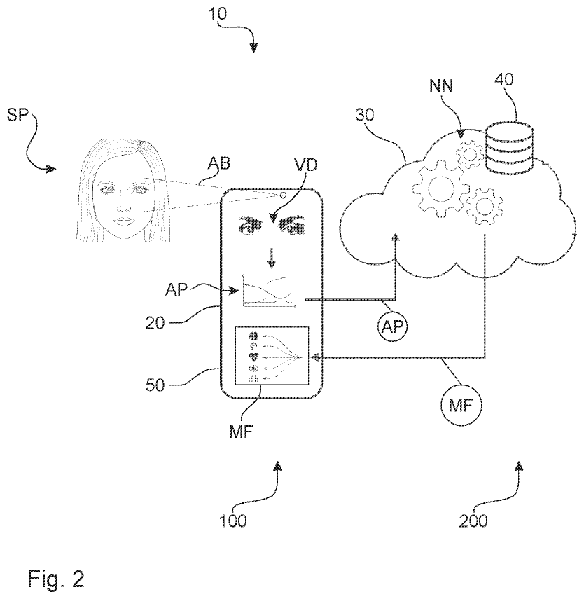

FIG. 2 an embodiment of an assignment system according to the invention,

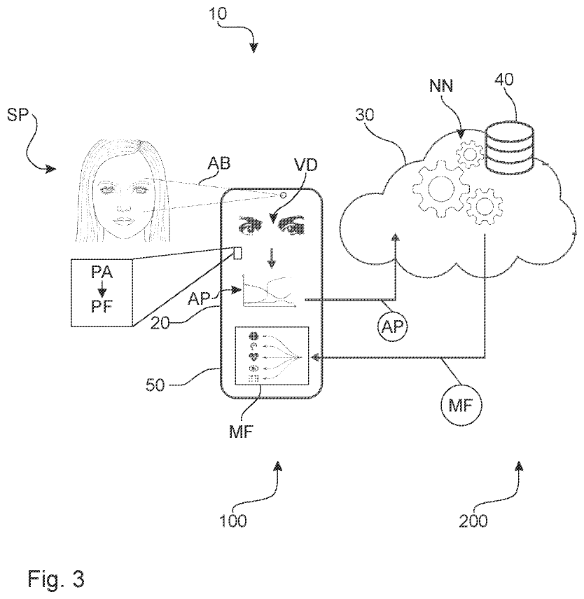

FIG. 3 an embodiment of an assignment system according to the invention,

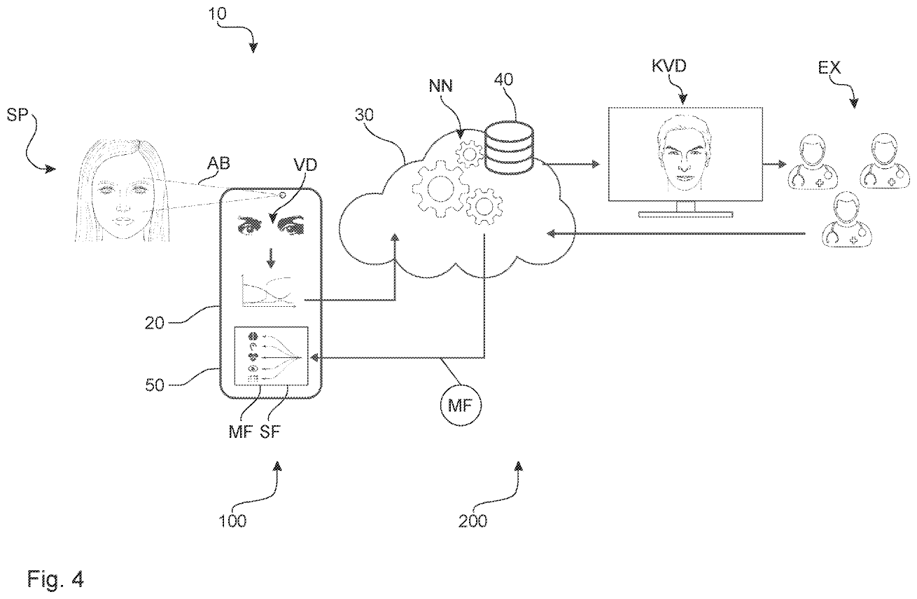

FIG. 4 an embodiment of an assignment system according to the invention,

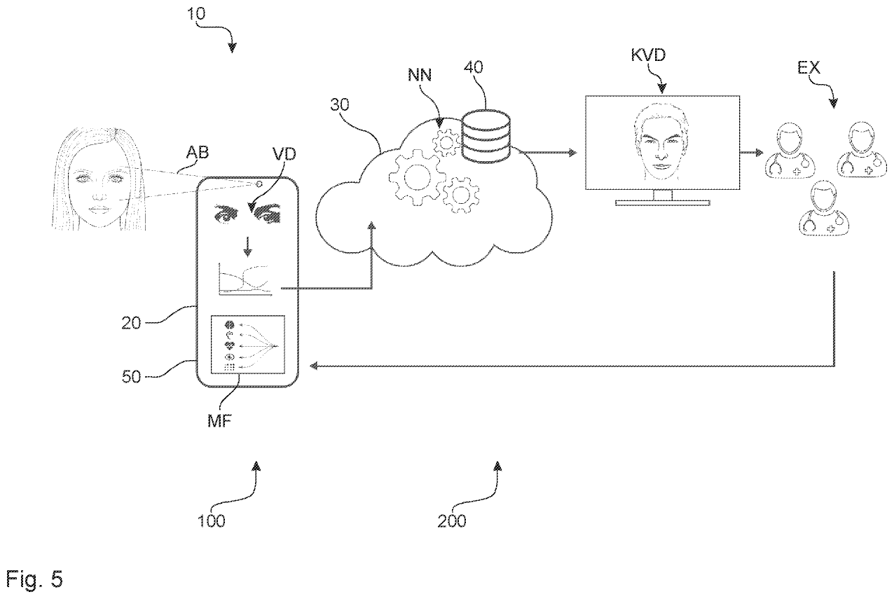

FIG. 5 an embodiment of an assignment system according to the invention,

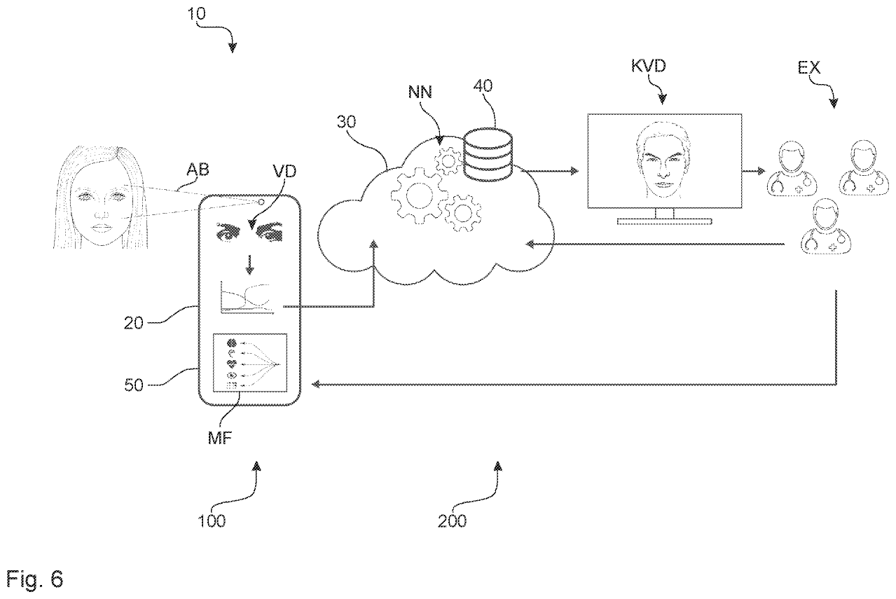

FIG. 6 an embodiment of an assignment system according to the invention,

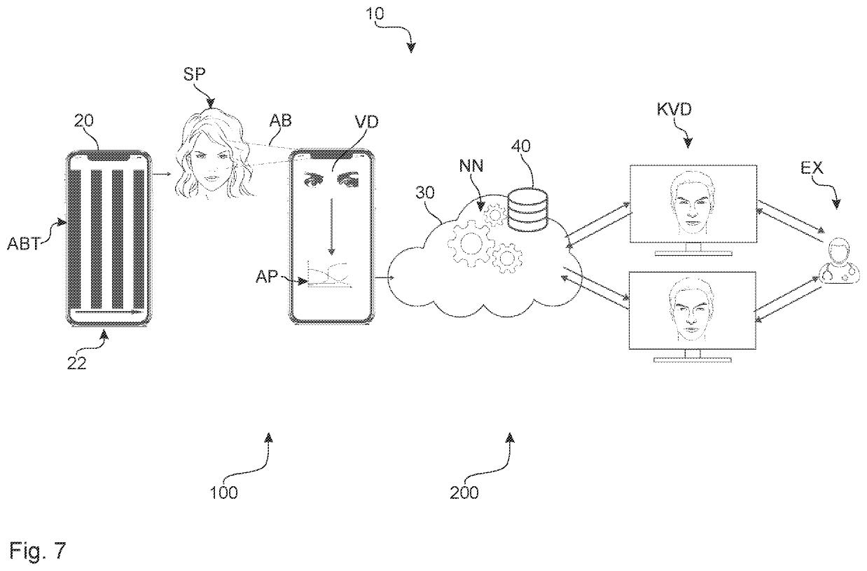

FIG. 7 an embodiment of an assignment system according to the invention,

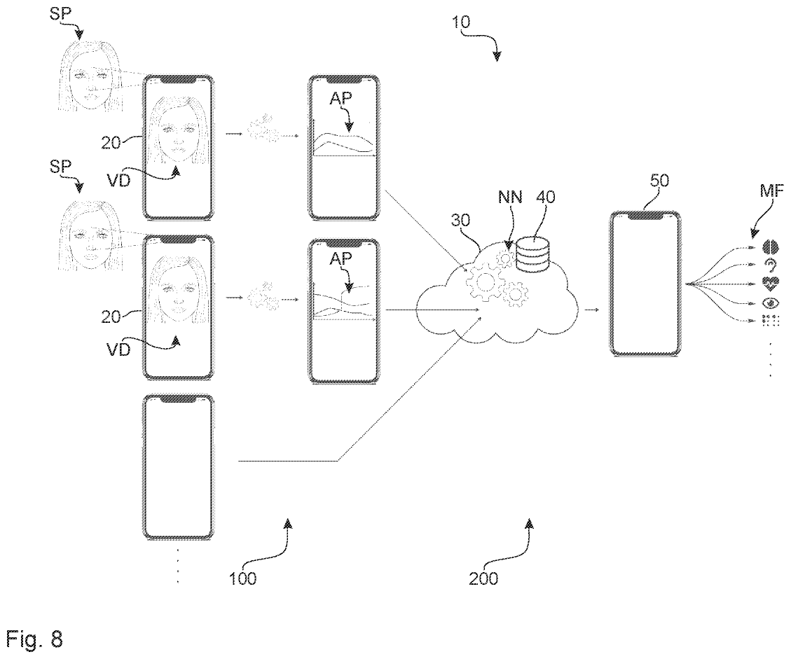

FIG. 8 an embodiment of an assignment system according to the invention,

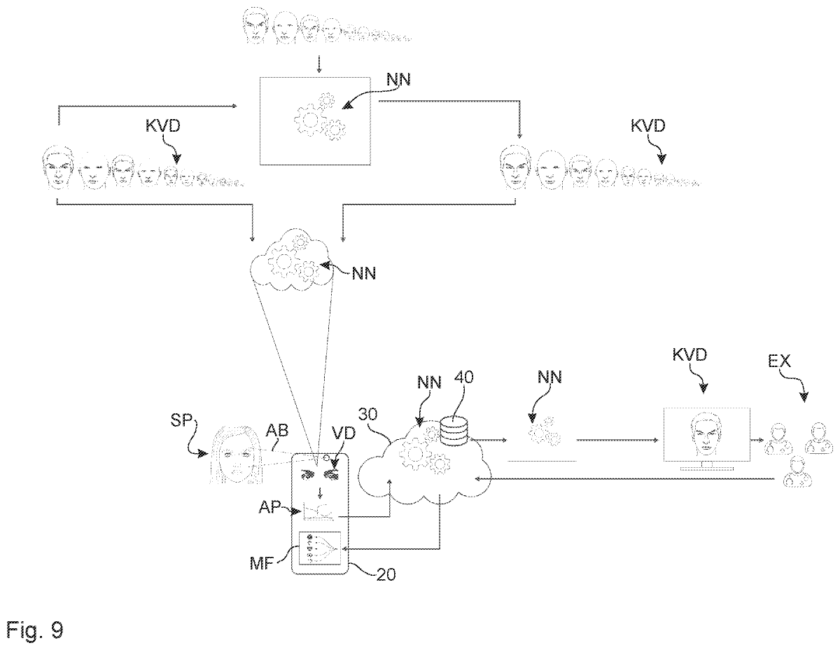

FIG. 9 an illustration of a training method according to the invention.

FIG. 1 shows the simplest configuration of an assignment system 10 according to the present invention. A separation into a local assignment device 100 and a central assignment device 200 can already be seen here. In principle, however, it would also be conceivable to carry out the entire procedure in the local assignment device 100.

As a local assignment device 100 of the assignment system 10, a smartphone is schematically shown here as an acquisition device 20. This is capable, with the aid of a camera, of recording the eye movements AB of a dizzy patient SP. This recording results in recorded eye movements AB in the form of video data VD. Subsequently, either locally or, as shown in FIG. 1, in a central assignment device 200, these video data VD are processed in a processing device 30 with the aid of a neural network NN. With the aid of the determination device 40, for example in the form of a cloud memory, an assignment of at least one medical specialty MF is made on the basis of the processing result, which is transmitted back to the mobile terminal as the local assignment device 100. The smartphone as the acquisition device 20 is also configured here as the output device 50 and is used to output the assigned medical specialty MF.

FIG. 2 shows a further development of the embodiment in FIG. 1. The decisive difference here is that the video data VD are no longer transmitted to the central assignment device 200 in the cloud. Instead, the video data VD is translated into eye movement parameters AP in the local assignment device 100, which represent an anonymization of the real video data VD. Thus, the transmission to the central assignment device is now performed in an anonymized manner or in a pseudonymized manner, if an assignment of the assignment is to be performed at the end of the procedure, by transmitting not the video data VD, but only the eye movement parameters AP. The further course of the processing and the determination as well as the output of the assignment is identical to the description of the embodiment of FIG. 1.

FIG. 3 shows another further development of the embodiment of FIG. 2. Here, additional patient questions PF are displayed on the output device 50 so that the dizzy patient SP can provide associated patient responses PA. These patient responses are entered, in particular, by the physician or health care provider accompanying the procedure, or by the patient himself. These patient responses are used to either select different eye movement tests ABT, to select different neural networks NN, or to perform a plausibility check of the assigned medical specialty MF during the output.

FIG. 4 is also a further embodiment of an assignment system 10. Here it is now possible, for example, for an assigned medical specialty MF to determine a safety factor SF with which probability it is the correct medical specialty MF. If the safety factor falls below a threshold value in particular, for example at a safety of less than 60 percent, in this embodiment the central assignment device 200 can allow a provision for a manual check by experts EX. A manual selection of a review by an expert EX is of course also conceivable. This can bring advantages in the execution of the method, as well as the training method. However, this provision does not take place directly, but in an anonymized manner, in that the anonymized data transmitted to the central assignment device 200 is transferred to a virtual image of a person in the form of artificial video data KVD. These artificial video data KVD can now be manually reviewed by the experts EX, so that, on the one hand, a reviewed output of the assigned medical specialty MF is possible and, on the other hand, this feedback serves a learning effect of the neural network NN.

FIG. 5 shows a variant of FIG. 4, whereby here in the form of telemedicine the verified assignment of the medical specialty MF is not made via the central assignment device 200, but directly from the experts EX to the local assignment device 100. In this case, videos are not sent to a medical expert EX as in other already known telemedical software approaches, but the extracted non-sensitive eye movements AB are inserted into an artificially generated head, so that artificial video data KVD are displayed in such a way as these medical experts EX are already accustomed to when directly examining patients, which thus increases the accuracy of assessment.

FIG. 6 shows the combination of the embodiment of FIGS. 4 and 5.

FIG. 7 shows schematically how an eye movement test ABT may proceed. Thus, a light-dark gradient/black-and-white bar pattern can be shown on the display device 22 of the acquisition device 10 in the form of a display, which in particular moves. Of course, other and more complex displays, such as cartoon characters or the like, are also conceivable within the scope of the present invention. The dizzy patient SP views this video of the eye movement test ABT, and during this viewing time his eye movements AB are recorded by the acquisition device 20. As a result of the video being played, certain eye movements, for example a saccade, are evoked in the viewer by continuously refocusing on a bar from the bar pattern, which provide information on a healthy or unhealthy behavior of the patient's eye movements. The subsequent sequence via processing in the neural network NN is again essentially identical in the central assignment device 200. Again, it is shown how even different artificial video data KVD can be generated, which can subsequently be viewed by one or more experts. This right-hand section of FIG. 7 serves in particular to enhance the learning effect or the training effect for the neural network NN. It is also shown that at least one expert has the possibility to manipulate the eye movements in such a way that in particular inconspicuous to conspicuous eye movements are specifically manipulated so that the training set can be provided with necessary data.

FIG. 8 shows how different video data VD can be recorded and subsequently anonymized for different eye movement tests ABT. In the middle it is shown how different eye movement parameters AP are generated for each eye movement test ABT, which can subsequently be processed either in a common neural network NN or in different neural networks NN in the processing device 30. The combination of these individual test results is now performed again on the output device 50 in the form of an assignment to one or more medical specialties MF.

FIG. 9 shows again how a training procedure can be performed in principle. During a normal run, real video data VD are generated from eye movements AB of the dizzy patient SP. In the manner already explained, these pass through the neural network NN and a downstream additional neural network NN to generate artificial video data KVD, which are then provided to the experts EX for manual labeling. This part is used to provide the labeled artificial video data KVD as training data to the neural network NN and in this way to adjust the weighting in the neural network NN for its training.

The upper section of FIG. 9 shows how a variety of training data can be generated from a single video recording. Thus, a video recording in the form of video data VD can be transferred by a neural network NN to a plurality of different virtual heads, so that many different artificial video data KVD can be generated from a real video recording. It is also conceivable that the artificial video data KVD are generated completely virtually at the top left, whereby the eye movements and further characteristics such as the head orientation or the like are known exactly by the virtual generation. These can be transferred, in particular starting from photorealistic artificial base representations, via a further neural network NN shown at the top center, into photorealistic artificial video data KVD at the top right, so that subsequently both the virtually generated artificial video data KVD and the photorealistic artificial video data KVD are used for training the neural network NN. Even with a relatively small data base, this results in a sufficient amount of training data being available with high accuracy and at high speed for training the neural network NN in the processing device 30.

The foregoing explanation of the embodiments describes the present invention exclusively in the context of examples. Of course, individual features of the embodiments may be freely combined with one another, provided that this is technically expedient, without departing from the scope of the present invention.

### LIST OF REFERENCE SIGNS

10 Assignment system
20 Acquisition device
22 Display device
30 Processing device
40 Determination device
50 Output device
100 Local assignment device
200 Central assignment device
SP Dizzy patient
MF Medical specialty
SF Safety factor
AB Eye movements
ABT Eye movement test
AP Eye movement parameter
VD Video data
KVD Artificial video data
PA Patient response
PF Patient question
NN Neural network
EX Experts

1. A method for assigning a dizzy patient to a medical specialty, comprising the following steps:
Capture of eye movements of the dizzy patient in the form of video data,
Processing the acquired video data in a neural network,
Determine at least one medical specialty based on the result of processing in the neural network,
Outputting an assignment of the dizzy patient to the at least one specific medical specialty.

2. Method according to claim 1, wherein the video data are anonymized before processing in the neural network, in particular translated into eye movement parameters.

3. Method according to claim 1, wherein the acquired video data are transmitted, in particular in anonymized form, from an acquisition device to a processing device in which the processing is carried out by means of the neural network, preferably the determined medical specialty being transmitted back to the acquisition device and output at the acquisition device by means of an output device.

4. Method according to claim 1, wherein at least one test video is played on a display device of an acquisition device during the capture of the eye movements.

5. Method according to claim 1, wherein, in addition to the video data, at least one patient response to at least one patient question is acquired.

6. Method according to claim 1, wherein the eye movements to at least two different eye movement tests are captured, wherein the video data to the different eye movement tests are processed in different neural networks.

7. Method according to claim 1, wherein a quality control of the video data is performed before and/or during and/or after the acquisition of the eye movements in the form of video data.

8. Method according to claim 1, wherein the determination of the at least one medical specialty includes a safety factor which includes the accuracy of the assignment of the at least one medical specialty.

9. Method according to claim 1, wherein the captured video data are made available for manual review.

10. Method according to claim 9, wherein the video data are made available in anonymized form, in particular as artificial video data for manual verification.

11. Method according to claim 1, wherein additional auxiliary parameters are recorded, in particular at least one of the following:
Brightness
Lighting situation
Accelerations of an acquisition device
Positioning of an acquisition device.

12. A training method for training a neural network for use in a method having the features of claim 1, comprising the following steps:
To provide a variety of eye movements of dizzy patients in the form of video data,
Manual labelling of the multitude of eye movements,
Training the neural network with the multitude of manually labelled eye movements.

13. Training method according to claim 12, wherein artificial video data are generated for it, which are manipulated in particular manually and/or automatically.

14. Training method according to claim 12, wherein the plurality of eye movements are at least partially labelled multiple times.

15. An assignment system for assigning dizzy patients to a medical specialty, comprising an acquisition device for capturing eye movements of the dizzy patient in the form of video data, a processing device for processing the detected video data in a neural network, a determination device for determining at least one medical specialty on the basis of the result of the processing in the neural network, and an output device for outputting an assignment of the dizzy patient to the determined at least one medical specialty, wherein the acquisition device, the processing device, the determination device and/or the output device are designed in particular for carrying out a method having the features of claim 1.

16. Assignment system according to claim 15, wherein the acquisition device and the output device are arranged in a local assignment device and at least the processing device is arranged in a central assignment device.

17. Computer program product comprising instructions that cause an assignment system having the features of claim 15 to perform a method for assigning a dizzy patient to a medical specialty, comprising the following:
Capturing the eye movements of the dizzy patient in the form of the video data,
Processing the acquired video data in the neural network,
Determining the at least one medical specialty based on the result of processing in the neural network,
Outputting the assignment of the dizzy patient to the at least one specific medical specialty.
